(19) United States
(12) Patent Application Publication
(10) Pub. No.: US 2020/0030014 A1
(43) Pub. Date: Jan. 30, 2020

(54) Positioning device for cranial bone portions, production method for positioning device and system comprising positioning device having fastening devices

(71) Applicant: Karl Leibinger Medizintechnik GmbH & Co. KG, Muhlheim (DE)

(72) Inventors: Frank Reinauer, Mühlheim (DE); Stefanie Grom, Mühlheim (DE); Anita Grigore, Mühlheim (DE); Veit Irion, Mühlheim (DE); Ernst-Johannes Haberl, Mühlheim (DE)

(73) Assignees: Karl Leibinger Medizintechnik GmbH & Co. KG, Mühlheim (DE); Karl Leibinger Medizintechnik GmbH & Co. KG, Mühlheim (DE)

(21) Appl. No.: 16/603,018

(22) PCT Filed: Apr. 4, 2018
(86) PCT No.: PCT/EP2018/058636

§ 371 Date: Oct. 4, 2019

(30) Foreign Application Priority Data
Apr. 4, 2017 (DE) 102017107259.4

(51) Int. Cl. A61B 17/88; A61B 17/68; A61B 17/80

(52) U.S. Cl. CPC A61B 17/8872; A61B 17/688; A61B 2017/568; A61B 17/8866; A61B 17/8061

(57) ABSTRACT

The invention relates to a positioning device (1) for cranial bone portions, for relative positioning of the cranial bone portions relative to each other and for obtaining a predefined overall shape and overall contour in order to achieve a target cranial shape, having a primary body (2) designed to bear the cranial bone portions, wherein the primary body (2) is produced based on individual patient data, which are specific for the patient to be treated, and supplemented by modified, anatomical data. The invention furthermore relates to a production method for producing such a positioning device (1), which is prepared to have cranial bone portions temporarily fastened to it, wherein in one manufacturing step, a primary body (2) of the positioning device (1) is produced, based on geometric data representative of the cranial bone geometry of the particular patient to be treated. The invention also relates to a system comprising a positioning device (1) according to the invention and a cranial-bone fastening device (16) for temporary fastening of a cranial bone portion to the primary body (2) of the positioning device (1) in order to orientate the cranial bone portions relative to each other, wherein a pin-shaped fixing element portion (17) is provided on the cranial-bone fastening device (16), which is designed to contact the cranial bone portion or several cranial bone portions on one side while interacting with a counter portion (18) arranged on the other side of the cranial bone portion or cranial bone portions to fasten it or them on the primary body (2) of the positioning device (1).

The invention relates to a positioning device for cranial bone portions for relative positioning of the cranial bone portions to each other and for obtaining a predefined overall shape and overall contour to achieve a target cranial shape, with a primary body designed to support the cranial bone portions. Furthermore, the invention relates to a production method for producing a positioning device and a positioning device to which one or more fastening devices are attached or can be attached for positioning cranial bone portions, wherein the fastening device(s) is/are configured to temporarily fasten the cranial bone portions to the positioning device.

In order to correct cranial deformations, the deformed cranial bone portions are usually brought into an undeformed shape by a surgical procedure.

From the prior art, devices for the orientation (and correction) of deformed cranial bone portions are already known. EP 2 522 289 A1, inter alia, discloses a three-dimensional, life-size model of a human skull comprising a forehead part, a vertex part, and an occiput part, wherein it is sterilizable. Such a positioning device is mainly used to allow correcting disorders of cranial growth. The deformed cranial bone portions are repositioned by means of surgery and adapted to a desired cranial shape.

However, the prior art always has the disadvantage that positioning devices for repositioning the cranial bone portions are used which represent the model of an average child's skull. However, even with a large selection of skull models, the individually desired skull only corresponds partially or largely to the average model. However, since the deviations of an individual skull from the next closest, average model are very large, no optimal result can be achieved when modeling the cranial bone portions. The success of the surgery then depends on the skill and ability of the surgeon.

It is therefore the object of the invention to avoid the disadvantages of the prior art or at least to mitigate them. In particular, a positioning device is to be developed that is optimally adapted to the respective individual skull, so that the modelling of the cranial bones is considerably facilitated, since the surgeon no longer has to adjust the cranial bones manually.

The object of the invention is solved according to the invention by producing the primary body of the positioning device based on individual patient data specific to the patient to be treated and supplemented by modified anatomical data, e.g. in a generic method step.

The target cranial shape is defined by calculating an average shape as well as most/all characteristic variations of the shape on the basis of geometric data from healthy, non-deformed skulls. Associated geometric points of each skull surface are determined by transferring/projecting/applying anatomically associated regions from one skull shape to another, minimizing metric deformations of deformed parts. This modified, anatomical data, which is obtained by adjusting the statistically detected, undeformed skull shapes to deformed parts of the skull, taking into account the variations of the skull shape, can thus be used to calculate an individual, desired skull shape, which adjusts and corrects the deformed parts of the skull in such a way that an undeformed skull shape is created. “Modification” is (also) understood to mean ‘further processing’. Thus, reference points are selected on a comparable, undeformed skull, determined as correction values, and then incorporated into the specific skull by further processing.

This has the advantage that the repositioned cranial bone portions can easily be brought into an individual, desired skull shape with the help of the positioning device. The removed cranial bone portions can therefore easily be placed on the outside or inside of the positioning device and can be adapted to its shape in order to achieve an optimal result. It is no longer necessary to manually perform individual adjustments and corrections of the deviations from a model based on an average skull, since the individual, desired skull shape/target cranial shape is already appropriately considered for the creation of the primary body of the positioning device. It is advantageous if the person-related patient data concerning the skull allow the individual, desired skull shape to be calculated.

The object of the invention is also solved according to the invention by using a positioning device for cranial bone portions for relative positioning of the cranial bone portions to each other and for obtaining a predefined overall shape and overall contour to achieve a target cranial shape, with a primary body designed to support the cranial bone portions, wherein the primary body has a circumferential edge and limits a carrying structure.

Advantageous embodiments are claimed in the dependent claims and are explained in further detail in the following.

It is also advantageous if the edge is designed to be closed, since this increases the stability of the positioning device. The edge thus serves as a base plate/base outline and as a limitation for the carrying structure to which the cranial bone portions are attached for modelling.

Furthermore, a favorable exemplary embodiment is distinguished by the fact that the carrying structure is arranged on only one side of an imaginary surface or plane running through the edge. This determines exactly in which area the cranial bone portions have to be arranged.

Furthermore, it is practical if the surface is predefined by a common intersection of several planes. Thus, the edge has an almost flat outer edge, which can also be used as orientation for a horizontal arrangement of the positioning device.

It is also advantageous if a handle part/holding element protrudes outwards from the primary body, approximately in the area of the edge and/or the carrying structure, in order to hold, fasten, and/or clamp the positioning device. This facilitates holding, in particular during surgery. It is also made possible that the positioning device can be fixed firmly so that it cannot tilt.

If the handle part/holding element is approximately or exactly perpendicular to the primary body measured at the joint contact point, then it is particularly convenient to clamp the handle part/holding element. In particular, the handle part/holding element can protrude in a horizontal direction, since this additionally improves the orientation for arranging the cranial bone portions.

It is also advantageous if the edge has an approximately constant thickness and/or constant height over the circumference so that stability over the circumference is constant and the tension in the edge is uniform. However, the thickness and/or the height can also be greater or smaller at some points of the edge distributed over the circumference, for example in the area of the holding element.

A favorable exemplary embodiment is also distinguished by the fact that the edge region has an elevation and/or a thickening in the area of the protruding handle part/holding element. Thus, the handle part/holding element can be connected to the primary body of the positioning device in a more stable way and thus guarantee a better force transmission.

Moreover, it is practical if the carrying structure is net-like or lattice-like, which advantageously lowers the weight of the carrying structure, which improves handling, and also makes it possible to reach through it with a fastening element to temporarily fix the cranial bone portions to the positioning device.

Furthermore, it is advantageous if the carrying structure has a lattice structure, net structure, or intersecting line-structure, which ensures optimized strength of the carrying structure at low weight.

The primary body can also be designed as a convex shell and/or it can have a preferably constant thickness. In particular, it is important that the side or surface, in particular the inside/inner surface, of the primary body, on which the cranial bone portions are placed, is convex, since it thus adapts to a typical skull shape. It is in particular advantageous if the primary body is approximately hemispherically shaped.

The primary body of the positioning device can be helmet-like, i.e. the inner side of the primary body adapts to the outer side of the skull. Thus, the cranial bone portions can be arranged on the inside and brought into the desired skull shape.

It is furthermore practical if the carrying structure has a large number of recesses or through-holes which are spatially separated from each other by webs. These recesses or through-holes thus advantageously allow penetration (for the fastening elements) through the carrying structure, which is particularly important for fixing the cranial bone portions to the carrying structure.

It is advantageous if the webs form a polygon shape, for example defining a regular polygon, such as a honeycomb structure and/or a hexagonal shape with, e.g., rims of equal length or a hole geometry with continuously rounded outer contour. Thus, the webs enable that part of the fastening element for fixing the cranial bone portions can rest on the carrying structure and another part of the fastening element can penetrate through the recesses.

Furthermore, it is practical if an outer contour of all or some through-holes corresponds in its geometry and/or surface. In this way, the same fastening elements can be used at different/all places on the carrying structure.

Furthermore, a favorable exemplary embodiment is distinguished by the fact that the through-holes have a uniform configuration/characteristic. Thus, it is not important in which orientation, in particular in which position the fastening elements are inserted through the through-holes when rotating around a direction protruding in the radial direction of the positioning device.

It is also possible, if the through-holes have a circular, rounded, elliptical, or oval surface, that they can safely hold advantageous embodiments of a fastening element adapted to it.

In addition, it is practical if the webs have a constant and/or uniform thickness. This ensures that there are no local weak points or tension peaks in the webs.

It is furthermore advantageous if the webs have a trapezoidal or triangular cross-section, which additionally supports the convex configuration of the carrying structure.

The webs can also have a quadrangular cross-section, wherein two main limiting surfaces run towards each other extending predominantly in the radial direction and two secondary limiting surfaces limiting these two main limiting surfaces are arranged parallel to each other. Thus, the webs form a larger surface on the inside of the carrying structure than on the outer surface, which advantageously offers a larger contact area for the cranial bone portions.

It is also advantageous if the two main limiting surfaces approach each other as seen from the inside to the outside, for example by forming an angle between 20° and 45°, for example 30°, 33° and 35°.

Moreover, it is practical if at least one or all of the webs have a length/width ratio of 10/3±10 percent and/or a length/thickness ratio of 10/3±10 percent. This ensures a favorable force transmission between the webs.

Basically, a favorable exemplary embodiment is distinguished by the fact that the edge has a height that is between 2 and 4 times as large as the thickness or width of the webs. This additional reinforcement at the edge of the carrying structure ensures that the shape is retained.

It is also advantageous if the positioning device has an inclination on the rear side. The rear side is the side on which the cranial bone portions, which normally lie on the occiput, are arranged. This inclination facilitates the handling and arrangement of the cranial bone portions on the inside of the positioning device.

It is particularly advantageous if the inclination stands out at an angle of 30 to 60°, preferably 40 to 50°, more preferably about 25°.

It is also practical if the positioning device has a center line marking. This allows the surgeon to easily see how the cranial bone portions are to be arranged, since he gains a better orientation.

It is also advantageous if the center line marking is formed by a web running from a first region of the edge to an opposite region of the edge or a recess running accordingly. Preferably, the center line marking runs from the front side to the rear side, so that the vertex line of the cranial bone portion is marked.

It is also practical if the primary body is completely or partially made of plastic. Plastic is particularly suitable for medical applications, since many plastics can be sterilized and used in many fields.

It is also preferred if the primary body consists entirely or partially of polyamide. Polyamide has particularly favorable properties in terms of costs and weight and is suitable for this application.

It is also advantageous if the positioning device has an anti-slip coating, since it can be gripped or handled more easily and unintentional slipping is effectively prevented.

The object of the invention is also solved by a production method according to the invention for producing a positioning device, which is prepared to have cranial bone portions temporarily fastened to it, wherein in one manufacturing step, a primary body of the positioning device is generated/created/produced, based on geometric data representative of the cranial bone geometry of the particular patient to be treated.

It is an advantage if the geometric data of the patient is detected in a reference step/measurement step preceding the production method and is optionally further processed.

Furthermore, geometric data can be acquired in the reference step by photometric means and/or by photometry, which advantageously avoids radiation exposure for the patient and is therefore particularly recommended for use with children.

In particular, cephalometric points are used as measuring points for the geometric data, since an entire, typical, desired cranial shape can be calculated from a few cephalometric points using a calculation formula. This means that it is no longer necessary to rely on average values or statistical evaluations, but rather that an individual, perfectly adapted, desired cranial shape can be calculated.

It is practical if at least one of the cephalometric points is measured from glabella, opisthocranium, eurion, orbitale, nasion, pogonion, gnation, menton, gonion, bregma, lambda, zygion, porion, mastoidale, basion, inion or vertex. These points serve as starting points for the calculation formula for calculating a desired cranial shape.

It is advantageous if a virtual, desired skull model (of the skull with a desired shape) is calculated/created on the basis of the geometric data. The desired skull model thus corresponds to a model of a skull that is not deformed.

It is furthermore advantageous if the virtual, desired skull model is used to shape the inner surface of the positioning device so that it exactly matches the outer surface of the desired skull. Thus, it is advantageously possible to shape a deformed skull portion into the desired shape by placing it on the positioning device. Consequently, it is advantageous if the positioning device is designed in such a way that the inner surface of the positioning device corresponds to an outer surface of a desired skull/target skull.

It is also expedient if geometric values representative for the virtual, desired skull model are converted into geometric production data by a transfer step, based on which the positioning device, i.e. the primary body and, for example, the handle part/holding element, is produced in one manufacturing step.

It is therefore advantageous to first detect the geometric data, then calculate the geometric values based on them, i.e. the desired skull shape, and then calculate the geometric production data corresponding to a ‘helmet’ or an inside of the primary body or an outside of the desired skull model. The generative positioning device is then created from this geometric production data.

It is also advantageous if the positioning device is manufactured by additive manufacturing, for example by 3D printing. Thus, any shape can be created/printed based on the geometric production data at acceptable costs within a very short time.

The object of the invention is also solved by such a positioning device together with a cranial-bone fastening device/fastening device, which is configured to temporarily fasten the cranial bone portions to the positioning device. It is advantageous if the cranial-bone fastening device/fastening device is configured to temporarily fasten a cranial bone portion to the primary body of the positioning device.

The fastening device preferably has a fixing element portion which is configured for one-sided, direct contacting of the cranial bone portion or of several cranial bone portions on one side in order to fasten it or them to the primary body when interacting with a counter portion on the other side of the cranial bone portion or of the cranial bone portions. The fixing element portion and the counter portion are configured to engage in each other in the fastening state.

The fastening device may be constructed in at least two parts, wherein a part of the fastening device forms the fixing element portion having a pin, wherein the fixing element portion is dimensioned to extend from one side of the cranial bone portion to the other side of the cranial bone portion in the fastening state, and wherein another part of the fastening device forms the counter portion configured to receive the fixing element portion in such a way that the cranial bone portion is fastened to the positioning device.

The counter portion has a handle region and a bracing region. The fixing element portion has a handle region, a support region, and a coupling region with a thread and a pin.

The object of the invention is also solved by a method according to the invention for treating a patient, such as a human child, in the area of the head.

It is advantageous if at first the geometric data of the patient is detected, then the geometric values for the virtual, desired skull model are calculated, and based on this the geometric production data for the positioning device are calculated.

It is advantageous to generate the positioning device in a generative procedure before the surgery. In this way, the surgery can be performed as quickly as possible.

In addition, it is practical if the deformed cranial bone portions are removed in one surgery step. It is also advantageous to arrange the removed cranial bone portions in the positioning device and to orientate them to each other so that they form an approximately closed surface.

It is practical to fasten the orientated cranial bone portions with one or more cranial-bone fastening devices/fastening devices to the primary body of the positioning device.

Then, the cranial bone portions brought into the adapted shape can be fixed to each other via an approximately strip-shaped implant so that they maintain their position. The cranial-bone fastening devices can then subsequently be detached from the positioning device and the cranial bone portions.

Subsequently, the cranial bone portions attached to each other can be taken out of the positioning device and then be implanted back into the skull.

The invention is explained in the following with the help of the drawings. These show:

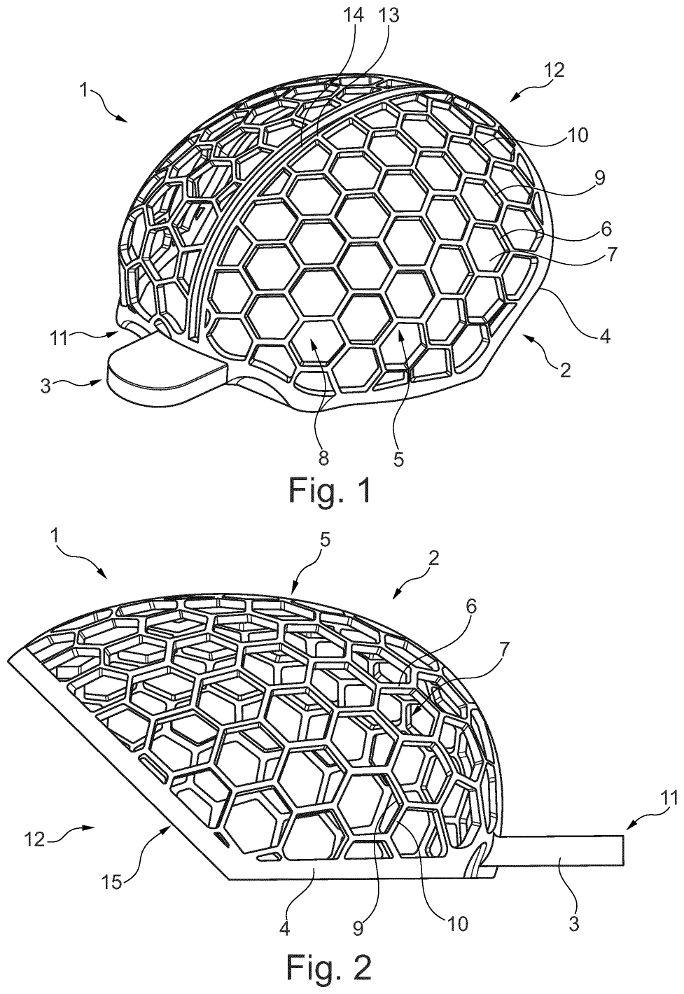

FIG. 1 shows a perspective view of a positioning device according to the invention with a lattice structure in a first exemplary embodiment and a center line marking,

FIG. 2 shows a side view of the positioning device in the first exemplary embodiment of the lattice structure,

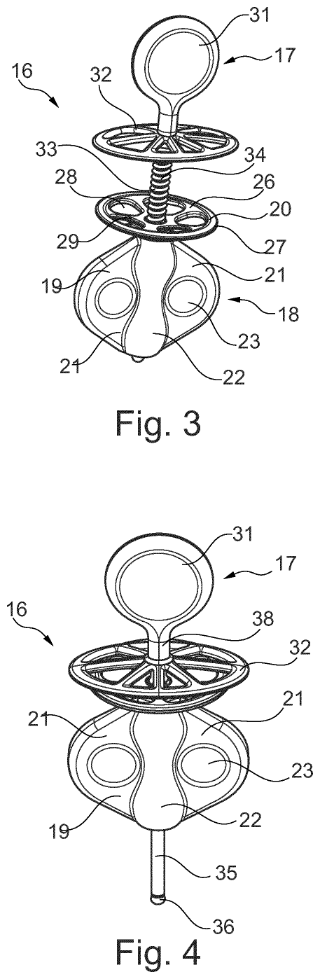

FIG. 3 shows a perspective view of a skull fastening device used for a system according to the invention with a counter portion and a fixing element portion,

FIG. 4 shows a perspective view of the cranial-bone fastening device with the fixing element portion screwed into the counter portion,

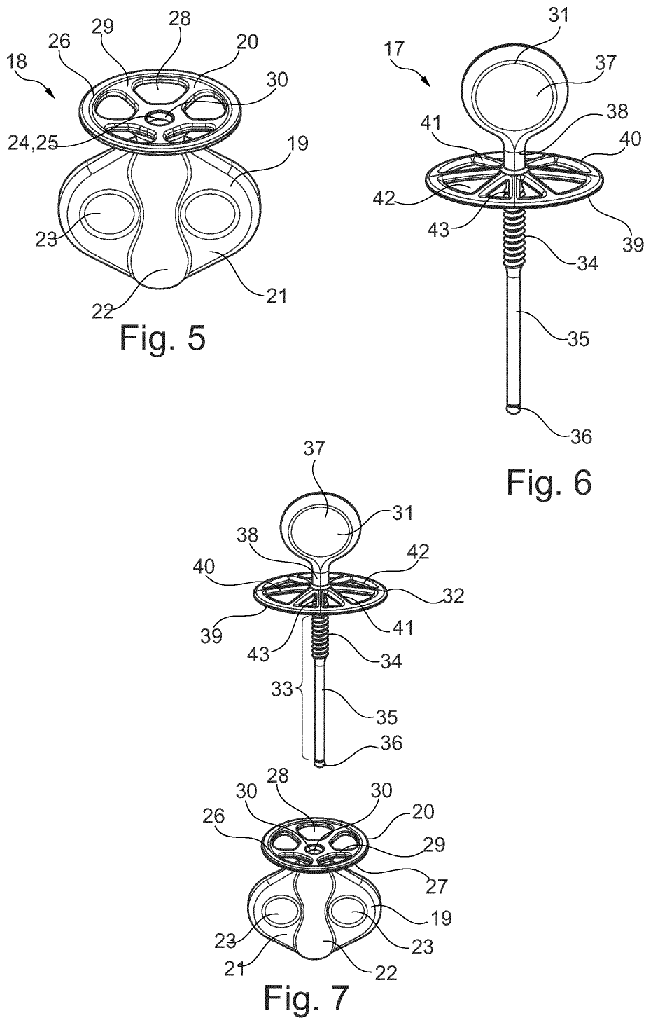

FIG. 5 shows a perspective view of the counter portion with a handle region and a bracing region,

FIG. 6 shows a perspective view of the fixing element portion with a handle region, a support region and a coupling region, and

FIG. 7 shows the skull fastening device in a perspective view.

The figures are merely schematic in nature and serve exclusively to understand the invention. The same elements are provided with the same reference signs.

FIG. 1 shows a positioning device 1 for cranial bone portions. The positioning device 1 is configured to position the cranial bone portions relative to each other and to maintain or create a predefined overall shape and overall contour of the cranial bone portions. The positioning device 1 has a primary body 2 on which the cranial bone portions are placed. Furthermore, the positioning device 1 has a handle part/holding element 3, which protrudes laterally, approximately at a right angle, from an edge 4 of the positioning device 1. The edge 4 is part of the primary body 2 and circumferentially limits a carrying structure 5, which is formed net-like/lattice-like and surrounds an imaginary hemispherical shape.

The carrying structure 5 is a convex shell and consists of webs 6 that limit several recesses/through-holes 7. The recesses have a hexagonal surface and form a kind of honeycomb shape 8. The recesses 7 are distributed evenly over the carrying structure 5 and are only separated from each other by the webs 6. The carrying structure 5 forms a round, approximately hemispherical shape. The carrying structure 5 extends only to one side of the edge 4, wherein the edge forms a circumferential, closed rim.

The webs 6 have a constant thickness and a square, in particular a trapezoidal cross-section. This means that the webs 6 have four surfaces, two of which are the main limiting surfaces 9 and two are secondary limiting surfaces 10. The main limiting surfaces 9 extend approximately in a radial direction of the positioning device 1 and approach each other from the inside to the outside. The secondary limiting surfaces 10, which extend in the circumferential direction, are arranged approximately parallel to each other.

A center line marking 13 extends from a front side 11 of the positioning device 1 to a rear side 12 of the positioning device 1. The center line marking 13 is a continuous recess 14 that extends from one side of the edge 4 to an opposite side of the edge 4. The front side 11 is the side of the positioning device 1 that would form the front side of the head if the positioning device 1 was placed over a skull like a helmet. Accordingly, the shape of the rear side 12 of the positioning device 1 corresponds to the occipital shape.

Following the recess 14 of the center line marking 13, the carrying structure 5 returns to its lattice-like/net-like/honeycomb-like structure with the webs 6 and the recesses 7. The carrying structure 5 forms an inclination 15 at the rear side 12, so that the carrying structure 5 does not enclose a complete hemisphere. In a side view (cf. FIG. 2) the edge 4 forms an obtuse angle within which the carrying structure 5 is arranged. The inclination 15 is positioned approximately at an angle of 45° to a horizontal.

At the front side 11 of the positioning device 1, the holding element 3 connects to the edge 4. The holding element 3 protrudes horizontally outwards from the primary body 2. The holding element 3 is formed with a constant thickness. The holding element 3 forms a surface, which is a rectangle with two rounded edges on the outer side. At the point of the edge 4 where the holding element 3 protrudes, the thickness/height of the edge 4 is increased as compared to the rest of the edge region. Overall, the primary body 2 is formed as a kind of convex shell with a thickness of about 2 to 5 mm.

The positioning device 1 is generated based on individual patient data by detecting comparative measurement points at the skull of the patient and calculating a virtual, desired cranial shape based on these points, which in turn are used to calculate geometric production data corresponding to the positioning device 1. The positioning device 1 is designed as a shell/helmet, which fits to the target cranial shape on the outside, so that the inside of the positioning device 1 can be used to adapt the shape of the cranial bone portions.

FIG. 2 shows a side view of the positioning device 1. Here it is particularly easy to see that the edge here partially extends in a horizontal direction and then bends upwards at an angle of about 45°.

FIG. 3 shows a cranial-bone fastening device 16 which is designed for temporary fastening of a cranial bone portion to the primary body 2 of the positioning device 1. The cranial-bone fastening device 16 has a fixing element portion 17 and a counter portion 18. The fixing element portion 17 is pin-shaped and is designed to contact the cranial bone portion or several cranial bone portions on one side while interacting with the counter portion 18 arranged on the other side of the cranial bone portion or cranial bone portions.

The counter portion 18 has a handle region 19 and a bracing region 20. The handle region 19 is plate-like and has two wings 21 which protrude from both sides of a thickening 22 in the same plane. A round, circular indentation 23 is formed on each wing 21. The two indentations 23 are arranged on both sides of the thickening 22 at the same distance. The thickening 22 is arranged in a longitudinal direction of the handle region 19. Inside the thickening 22, a recess 24 is arranged longitudinally in the manner of a through bore 25.

The thickening 23 is of non-constant thickness so that it has a larger diameter in the two edge regions arranged in the longitudinal direction than in the middle region arranged in the longitudinal direction. The handle region 19 of the counter portion 18 has a coating that acts in a particularly anti-slip, i.e. slip-resistant, manner. The handle region 19 of the counter portion 18 transitions into the bracing region 20 of the counter portion 18.

The bracing region 20 extends approximately plate-like into a plane which is orthogonal to the longitudinal direction of the counter portion 18. The bracing region 20 is thus formed in such a way that it has a concave surface 26 and a convex surface 27, which together form a convex shell of the bracing region 20. The convex surface 27 of the bracing region 20 is the side facing the handle region 19. The concave surface 26, on the other hand, is the surface of the bracing region 20, which is facing away from the handle region 19, i.e. facing the fixing element portion 17 and in the mounted state the cranial bone portion or the positioning device 1.

The bracing region 20 is formed to be round, in particular oval, and has recesses 28, which are separated from each other by webs 29. An inside thread 30 is integrated in the through bore 25 within the thickening 22 of the handle region 19 and the bracing region 20. The inside thread 30 can extend over the entire length of the through bore 25 or only over a section of the through bore 25.

The counter portion 18 of the cranial-bone fastening device 16 is engaged by the fixing element portion 17 in the mounted state. The fixing element portion 17 consists of a handle region 31, which transitions via a web 38 into a support region 32, wherein the support region 32 is followed by a coupling region 33. The coupling region 33 consists of a thread region 34 and a threadless end pin 35, which ends with a thickening 36. The thread region 34 has an outside thread, which is adjusted to the counter portion 18 in such a way that the outside thread of the thread region 34 can engage with the inside thread 30 in the through bore 25.

FIG. 4 shows the cranial-bone fastening device 16 in a state in which the fixing element portion 17 with its thread region 34 is screwed into the inside thread 30 of the counter portion 18. The cranial bone portion is fixed to the positioning device 1 between the bracing region 20 of the counter portion 18 and the support region 32 of the fixing element portion 17 during use.

FIG. 5 shows an enlarged view of the counter portion 18. Here, it is particularly easy to see that the inside thread 30 is formed in the through bore 25. The counter portion 18 is arranged either on the inside of the positioning device 1 or on the outside of the positioning device 1. Since the cranial bone portions are arranged on the inside of the positioning device 1, the counter portion 18 contacts either the cranial bone portions on the inside, in particular with its bracing region 20, or the lattice structure/carrying structure 5 of the positioning device 1 on the outside.

FIG. 6 shows an enlarged view of the fixing element portion 17. The handle region 31, which has a central indentation 37, transitions into the support region 32 via the small web 38. The support region 32 is formed like an oval plate. Equivalent to the bracing region 20, the support region 32 has a concave surface 39, which is arranged on the side facing the coupling region 33 and is configured to contact the cranial bone portion or the positioning device 1, and has a convex surface 40, which is arranged on the side facing the handle region 31. The support region 32 is formed by webs 41, which separate several approximately triangular recesses 42 from each other.

The coupling region 33 is connected centrally to the support region 32. Between the support region 32 and the thread region 34, there is a thinned section of the coupling region 33, which serves as a predetermined breaking point 43 for protection against incorrect use. The predetermined breaking point 43 ensures that the coupling region 33 breaks off from the support region 32 when an excessive force is applied in the axial direction or in a direction orthogonal or inclined to the axial direction. The predetermined breaking point 43 is adjoined by the thread region 34, which is matched to the inside thread 30 of the counter portion 18.

The thread region 34 transitions into a threadless end pin 35. The end pin 35 is matched to the counter portion 18 in such a way that the length of the end pin 35 is at least as large as the length of the entire counter portion 18 in the longitudinal direction. This has the purpose that the end pin 35 can be passed through the through bore 25 of the counter portion 18 without the inside thread 30 and the thread region 34 interlocking. The thickening 36 is arranged at a distal end of the end pin 35. The through bore 25 has a larger diameter than the thickening 36. However, the through bore 25 has a tapering at an axial end (or at any point in the through bore 25), so that the through bore 25 has a smaller diameter in the area of the tapering than the thickening 36 of the fixing element portion 17. The thickening 36 thus serves as a loss protection. When inserting the fixing element portion 17, the thickening 36 of the fixing element portion 17 is elastically deformed in such a way that the thickening can be passed through the tapering/tapered section in the through bore 25. The thickening 36 thus prevents the fixing element portion 17 from slipping back through the through bore 25 of the counter portion 18 without any force, i.e. unintentionally.

FIG. 7 shows the two portions 17, 18 of the cranial-bone fastening device 16 in a state in which they do not interact. For fastening the fixing element portion 17, it is pushed through the through bore 25 of the counter portion 18 with its coupling region 33. The cranial bone portions to be fastened are clamped together with the positioning device 1 between the support region 32 and the bracing region 20. The coupling region 33 can either be passed through the cranial bone portions or it can be arranged between several cranial bone portions. The coupling region thus engages the counter portion 18 in such a way that the cranial bone portions are attached via the bracing region 20 and the support region 32, since they are held between the bracing region 20 and the support region 32 at the positioning device 1.

### LIST OF REFERENCE SIGNS

1 positioning device
2 primary body
3 holding element
4 edge
5 carrying structure
6 web
7 recess/through-hole
8 honeycomb shape
9 main limiting surface
10 secondary limiting surface
11 front side
12 rear side
13 center line marking
14 recess
15 inclination
16 cranial-bone fastening device
17 fixing element portion
18 counter portion
19 handle region
20 bracing region
21 wings
22 thickening
23 indentation
24 recess
25 through bore
26 concave surface
27 convex surface
28 recess
29 web
30 inside thread
31 handle region
32 support region
33 coupling region
34 thread region
35 end pin
36 thickening
37 indentation
38 web
39 concave surface
40 concave surface
41 web
42 recess
43 predetermined breaking point

1. Positioning device (1) for cranial bone portions for relative positioning of the cranial bone portions to each other and for obtaining a predefined overall shape and overall contour to achieve a target cranial shape, with a primary body (2) designed to support the cranial bone portions, characterized in that the primary body (2) is created based on representatively individual patient data specific for the patient to be treated and supplemented by modified anatomical data, which is obtained by adjusting the statistically detected, undeformed skull shapes to deformed parts of the skull, taking into account the variations of the skull shape.

2. Positioning device (1) for cranial bone portions for relative positioning of the cranial bone portions to each other and for obtaining a predefined overall shape and overall contour to achieve a target cranial shape, with a primary body (2) designed to support the cranial bone portions, preferably according to claim 1, characterized in that the primary body (2) has a circumferential edge (4) and limits a carrying structure (5).

3. Positioning device (1) according to claim 2, characterized in that the carrying structure (5) has a net-like or lattice-like shape.

4. Positioning device (1) according to one of claim 2 or 3, characterized in that a holding element (3) protrudes outwards from the primary body (2) in order to hold, fasten, and/or clamp the positioning device (1).

5. Positioning device (1) according to claim 4, characterized in that the positioning device (1) has an inclination (15) on a rear side.

6. Positioning device (1) according to one of claims 2 to 5, characterized in that the primary body (2) is formed as a convex shell.

7. Positioning device (1) according to one of claims 2 to 6, characterized in that the carrying structure (5) has a plurality of recesses (7) or through-holes (7) which are spatially separated from each other by webs (6).

8. Positioning device (1) according to one of claims 2 to 7, characterized in that the positioning device (1) has a center line marking (13).

9. Production method for producing a positioning device (1), which is prepared to have cranial bone portions temporarily fastened to it, wherein in one manufacturing step, a primary body (2) of the positioning device (1) is produced, based on geometric data representative of the cranial bone geometry of the particular patient to be treated, and supplemented by modified anatomical data, which is obtained by adjusting the statistically detected, undeformed skull shapes to deformed parts of a skull, taking into account the variations of the skull shape.

10. System comprising a positioning device (1) according to one of claims 1 to 8 and a cranial-bone fastening device (16) for temporary fastening of a cranial bone portion to the primary body (2) of the positioning device (1) in order to orientate the cranial bone portions relative to each other, characterized in that a pin-shaped fixing element portion (17) is provided on the cranial-bone fastening device (16), which is designed to contact the cranial bone portion or several cranial bone portions on one side while interacting with a counter portion (18) arranged on the other side of the cranial bone portion or cranial bone portions to fasten it or them on the primary body (2) of the positioning device (1).
